(19) United States
(12) Patent Application Publication
(10) Pub. No.: US 2021/0007677 A1
(43) Pub. Date: Jan. 14, 2021

(54) DIAGNOSTIC ASSISTANCE SYSTEM, DIAGNOSTIC ASSISTANCE METHOD, AND DIAGNOSTIC ASSISTANCE PROGRAM

(71) Applicant: TERUMO KABUSHIKI KAISHA, Tokyo (JP)

(72) Inventors: Shin MAKI, Ebina-shi (JP); Tomoko UEMURA, Hadano-shi (JP)

(73) Assignee: TERUMO KABUSHIKI KAISHA, Tokyo (JP)

(21) Appl. No.: 17/032,851

(22) Filed: Sep. 25, 2020

Related U.S. Application Data
(63) Continuation of application No. PCT/JP2019/012811, filed on Mar. 26, 2019.

(30) Foreign Application Priority Data
Mar. 26, 2018 (JP) 2018-058053

(51) Int. Cl. A61B 5/00; A61B 5/0205; A61B 5/103; A61B 5/02

(52) U.S. Cl. CPC A61B 5/742; A61B 5/02055; A61B 5/1118; A61B 5/02028; A61B 5/1032

(57) ABSTRACT

A diagnostic assistance system is disclosed for assisting in diagnosis of heart failure, the diagnostic assistance system including a data obtaining section configured to obtain measurement data on an amount of congestion in at least a part of a body of a patient and measurement data on a parameter related to a blood flow rate of the patient, and a display control section configured to display, on a display unit, a graph in which a first axis indicates the amount of congestion and a second axis indicates the parameter related to the blood flow rate. The display control section is configured to display, in the graph, a point corresponding to the measurement data on the amount of congestion and the measurement data on the parameter related to the blood flow rate.

### CROSS-REFERENCES TO RELATED APPLICATIONS

This application is a continuation of International Application No. PCT/JP2019/012811 filed on Mar. 26, 2019, which claims priority to Japanese Application No. 2018-058053 filed on Mar. 26, 2018, the entire content of both of which is incorporated herein by reference.

### FIELD OF THE INVENTION

The present disclosure generally relates to a diagnostic assistance system, a diagnostic assistance method, and a diagnostic assistance program configured to assist in diagnosing heart failure.

### BACKGROUND DISCUSSION

Heart failure refers to a disease in which the pump function of a heart is decreased, causing a decrease in cardiac output, congestion in lungs and systemic veins, and the like. A patient suffering heart failure often repeats rehospitalization because the heart failure may remit once but deteriorate gradually.

As a method of diagnosing such a heart failure, a Nohria-Stevenson classification (see “2013 ACCF/AHA Guideline for THE Management of Heart Failure,” [online], the American College of Cardiology Foundation (ACCF), the American Heart Association (AHA), [retrieved on Feb. 20, 2018], the Internet <URL: http://circ.ahajournals.org/content/128/16/e240>) is known which classifies the disease state of the heart failure into four states. In a diagnostic method using the Nohria-Stevenson classification, a doctor determines the presence or absence of congestion in the body of a patient and the presence or absence of hypoperfusion (whether or not blood can be sufficiently pumped through the body) from physical observation, and classifies the disease state of the patient.

A diagnosis using the Nohria-Stevenson classification is made on the basis of the observation of each doctor and thus, depends, for example, on the experience of the doctor. Therefore, in a case where a general physician at a clinic observes the progress of a patient who has achieved remission after receiving a treatment from a medical specialist in heart failure, and makes the patient see the medical specialist as appropriate according to the condition of the patient, there is no common index in diagnosis between the general physician and the medical specialist. Cooperation between the general physician and the medical specialist is therefore difficult to make. Thus, in diagnosis based on the Nohria-Stevenson classification, cooperation is difficult to make between doctors or the like.

### SUMMARY

A diagnostic assistance system, a diagnostic assistance method, and a diagnostic assistance program that can provide a common index for doctors or the like in diagnosis of heart failure on the basis of the Nohria-Stevenson classification.

In accordance with an aspect, diagnostic assistance system is disclosed for assisting in diagnosis of heart failure, the diagnostic assistance system including a data obtaining section configured to obtain measurement data on an amount of congestion in at least a part of a body of a patient and measurement data on a parameter related to a blood flow rate of the patient, and a display control section configured to display, on a display unit, a graph in which a first axis indicates the amount of congestion and a second axis indicates the parameter related to the blood flow rate. The display control section is configured to display, in the graph, a point corresponding to the measurement data on the amount of congestion and the measurement data on the parameter related to the blood flow rate.

In accordance with another aspect, a diagnostic assistance method is disclosed for assisting in diagnosis of heart failure, the diagnostic assistance method including obtaining measurement data on an amount of congestion in at least a part of a body of a patient and measurement data on a parameter related to a blood flow rate of the patient, and displaying a point corresponding to the measurement data on the amount of congestion and the measurement data on the parameter related to the blood flow rate in a graph in which a first axis indicates the amount of congestion and a second axis indicates the parameter related to the blood flow rate.

In accordance with an aspect, a non-transitory computer readable medium for assisting in diagnosis of heart failure is disclosed, the non-transitory computer readable medium having instructions operable to cause one or more processors to perform operations comprising: obtaining measurement data on an amount of congestion in at least a part of a body of a patient and measurement data on a parameter related to a blood flow rate of the patient, and displaying a point corresponding to the measurement data on the amount of congestion and the measurement data on the parameter related to the blood flow rate in a graph in which a first axis indicates the amount of congestion and a second axis indicates the parameter related to the blood flow rate.

According to an exemplary embodiment of the present disclosure, users can use the graph displaying the point corresponding to the measurement data on the amount of congestion in at least a part of the body of the patient and the measurement data on the parameter related to the blood flow rate of the patient as a common index in diagnosis of heart failure on the basis of the Nohria-Stevenson classification.

### BRIEF DESCRIPTION OF THE DRAWINGS

FIG. 1 is a diagram depicting an outline of a diagnostic assistance system according to an exemplary embodiment of the present disclosure.

FIG. 2 is a diagram depicting a Nohria-Stevenson classification table.

FIG. 3 is a block diagram depicting a hardware configuration of a server included in the diagnostic assistance system according to the exemplary embodiment of the present disclosure.

FIG. 4 is a block diagram depicting a functional configuration of a CPU of the server included in the diagnostic assistance system according to the embodiment of the present disclosure.

FIG. 5A is a diagram of assistance in explaining data handled by the diagnostic assistance system according to the exemplary embodiment of the present disclosure.

FIG. 5B is a diagram of assistance in explaining data processing of the diagnostic assistance system according to the exemplary embodiment of the present disclosure.

FIG. 6A is a diagram of assistance in explaining a graph displayed by the diagnostic assistance system according to the exemplary embodiment of the present disclosure.

FIG. 6B is a diagram of assistance in explaining a graph displayed by the diagnostic assistance system according to the exemplary embodiment of the present disclosure.

FIG. 7 is a flowchart depicting a diagnostic assistance method according to the exemplary embodiment of the present disclosure.

FIG. 8 is a flowchart depicting a diagnostic assistance method according to a modification of the exemplary embodiment.

FIG. 9A is a diagram of assistance in explaining a graph displayed by a diagnostic assistance system according to the modification of the exemplary embodiment.

FIG. 9B is a diagram of assistance in explaining a graph displayed by the diagnostic assistance system according to the modification of the exemplary embodiment.

### DETAILED DESCRIPTION

Set forth below with reference to the accompanying drawings is a detailed description of embodiments of a diagnostic assistance system, a diagnostic assistance method, and a diagnostic assistance program configured to assist in diagnosing heart failure representing examples of the inventive diagnostic assistance system, diagnostic assistance method, and diagnostic assistance program. Note that since embodiments described below are preferred specific examples of the present disclosure, although various technically preferable limitations are given, the scope of the present disclosure is not limited to the embodiments unless otherwise specified in the following descriptions. In the description of the drawings, same elements are identified by the same reference numerals, and repeated description of the same elements that are identified by the same reference numerals will be omitted. In addition, dimensional ratios of the drawings may be exaggerated for the convenience of description, and may be different from actual ratios.

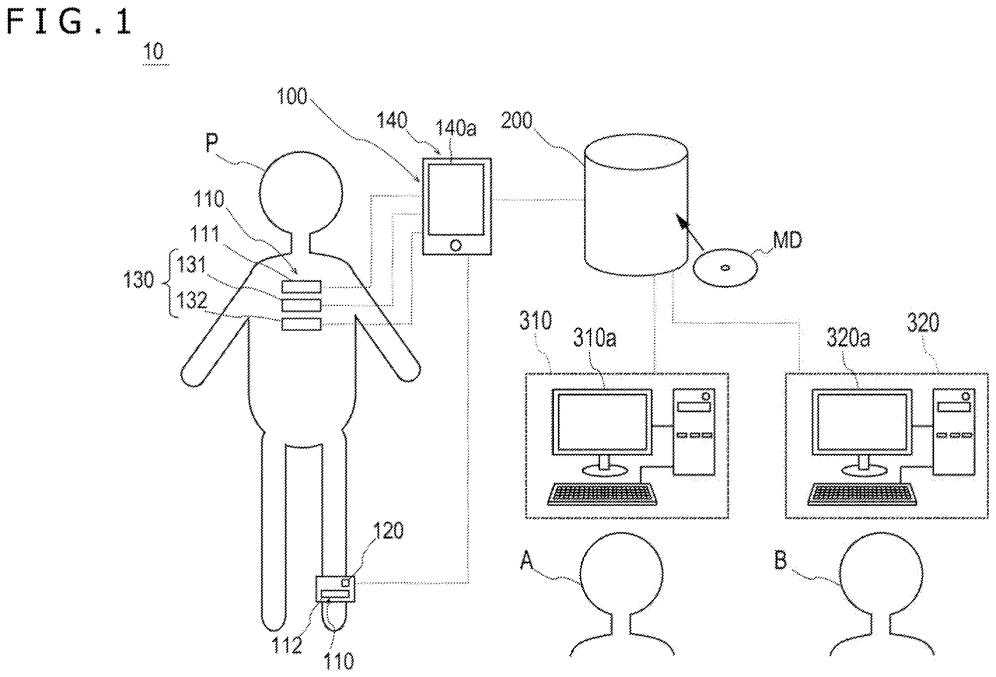

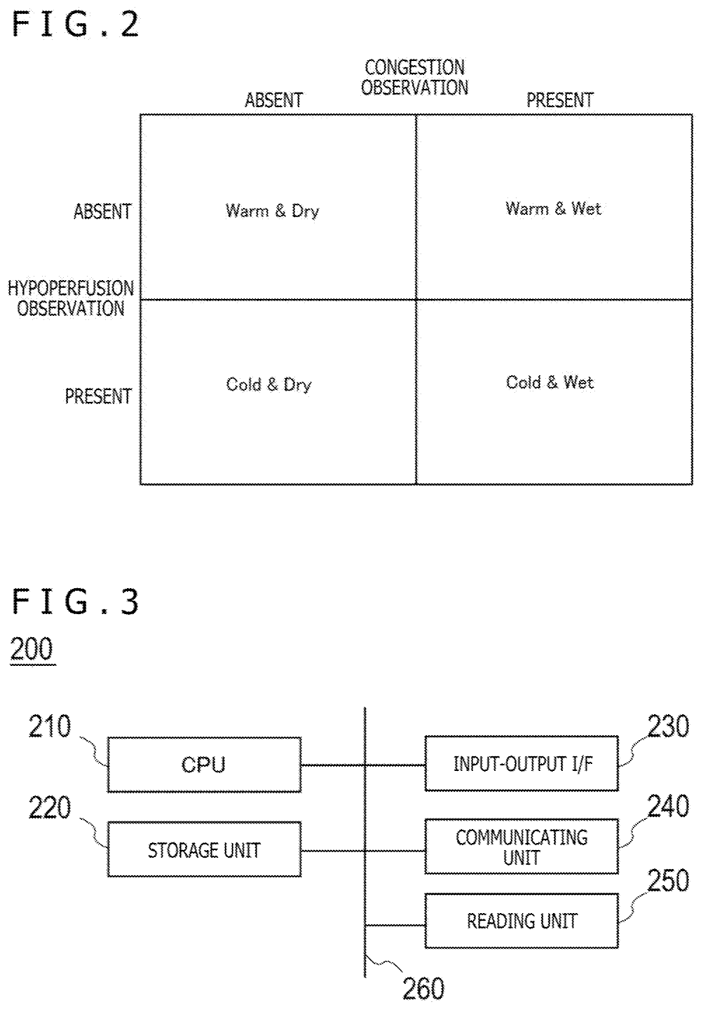

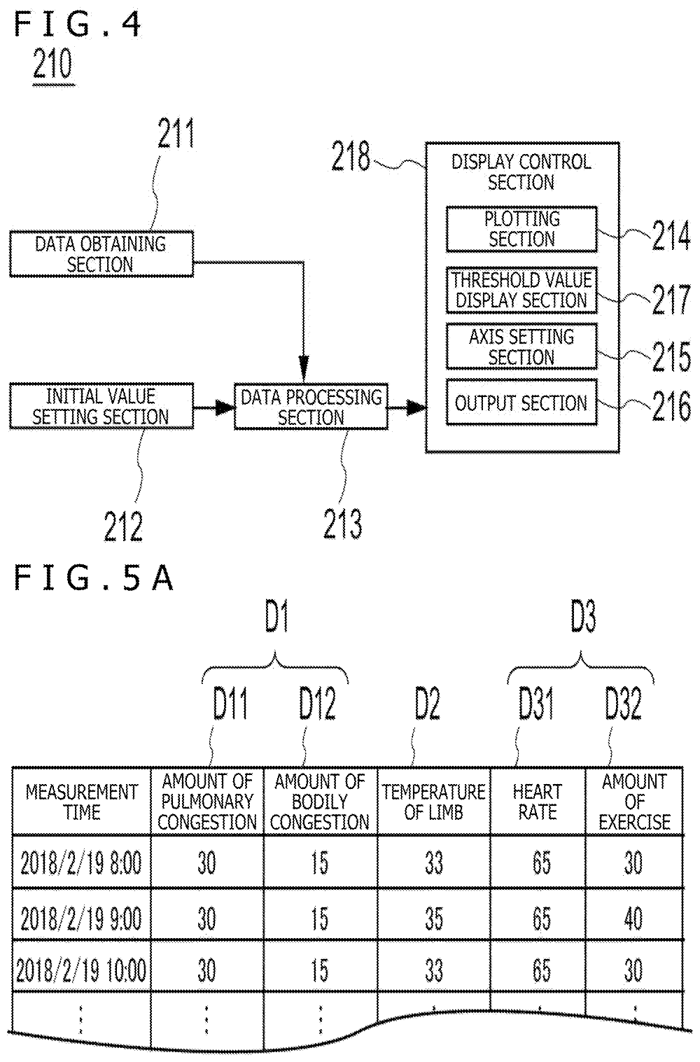

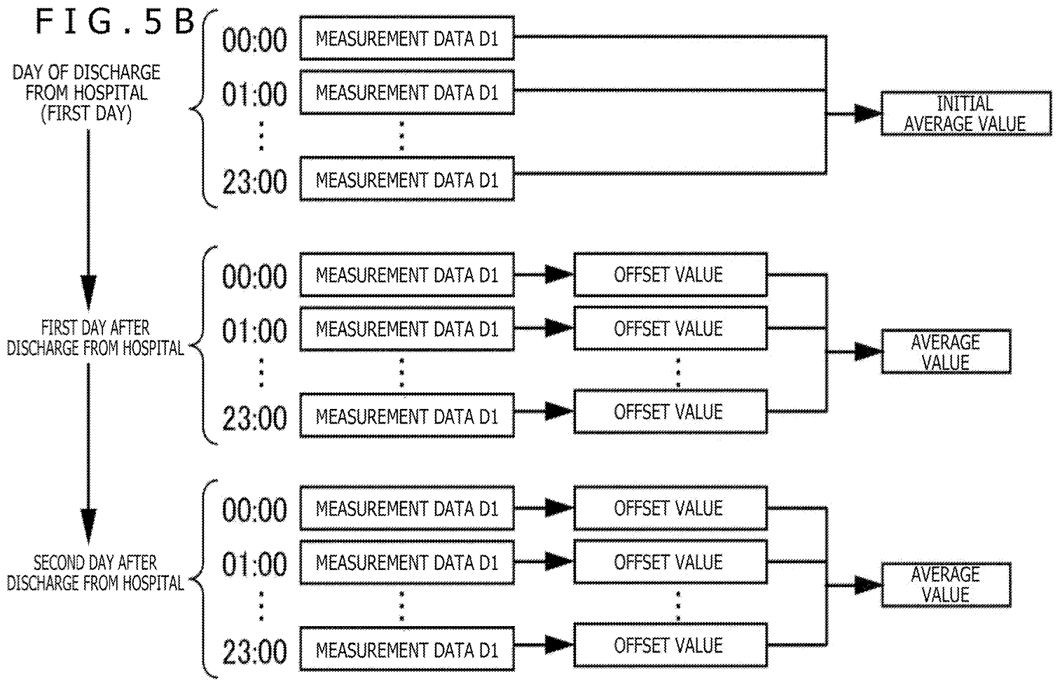

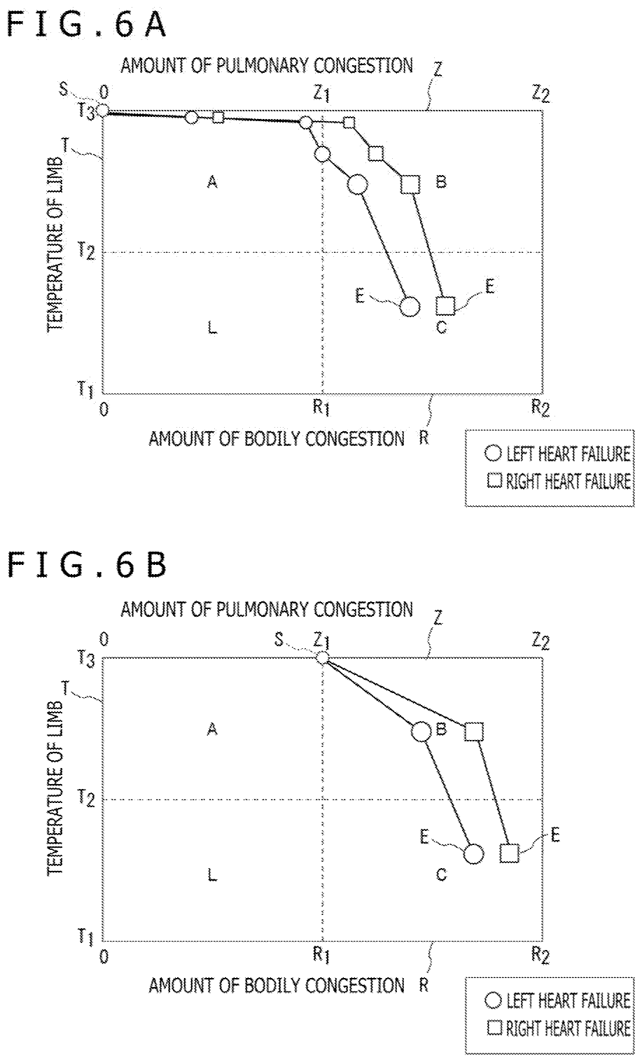

FIG. 1 is a diagram of assistance in explaining a general configuration of a diagnostic assistance system 10 according to the present exemplary embodiment. FIG. 2 is a diagram of assistance in explaining the Nohria-Stevenson classification. FIG. 3 and FIG. 4 are diagrams of assistance in explaining each part of the diagnostic assistance system 10. FIGS. 5A to 6B are diagrams of assistance in explaining data handled by the diagnostic assistance system 10.

As depicted in FIG. 1, in the present embodiment, the diagnostic assistance system 10 is configured as a system that can provide a plurality of doctors A and B as users of the diagnostic assistance system with information to be used to make a heart failure diagnosis based on the Nohria-Stevenson classification. Specifically, though not particularly limited, the diagnostic assistance system 10 can be, for example, used when a general physician B at a clinic observes the progress of a patient P who has achieved remission after receiving a treatment from a medical specialist A for heart failure, and recommends the patient P receive a treatment from the medical specialist A as appropriate according to the condition of the patient P.

As depicted in FIG. 2, the Nohria-Stevenson classification classifies the disease state of heart failure into four states on the basis of the presence or absence of congestion in the body of the patient P and the presence or absence of hypoperfusion (whether or not blood can be pumped through the body). A first disease state is Warm & Dry without congestion nor hypoperfusion (upper left in FIG. 2). A second disease state is Warm & Wet with congestion and without hypoperfusion (upper right in FIG. 2). A third disease state is Cold & Dry without congestion and with hypoperfusion (lower left in FIG. 2). A fourth disease state is Cold & Wet with congestion and with hypoperfusion (lower right in FIG. 2). Warm & Dry is a good state as a condition of the patient P. Warm & Wet, Cold & Dry, and Cold & Wet (Cold & Wet, in particular) are deteriorated states as conditions of the patient P.

Generally described with reference to FIG. 1, the diagnostic assistance system 10 includes a measuring unit 100 that measures an amount of congestion in at least a part of the body of the patient P and a parameter related to a blood flow rate of the patient P, and a server 200 that is connected to the measuring unit 100 and operating terminals 310 and 320 of the doctors A and B via a network (indicated by broken lines in the figure), and that transmits and receives data between the measuring unit 100 and the operating terminals 310 and 320. Each part of the diagnostic assistance system 10 will be described in detail in the following.

### Measuring Unit

The measuring unit 100 includes a congestion measuring part 110 capable of measuring an amount of congestion in at least a part of the body of the patient P, a blood flow rate measuring part 120 capable of measuring the parameter related to the blood flow rate of the patient P, a pump function measuring part 130 capable of measuring a parameter used in evaluation of a pump function of the heart of the patient P, and a control unit 140 that controls operation of these units. Each part of the measuring unit 100 will be described in detail in the following.

The measuring parts 110, 120, and 130 in the present exemplary embodiment are each formed by a wearable apparatus and are attached to the body of the patient P to perform measurement in a predetermined timing. The timing in which each of the measuring parts 110, 120, and 130 performs measurement is not particularly limited. However, each of the measuring parts 110, 120, and 130 can, for example, perform a measurement at intervals of one minute to one hour in a state in which the patient P wears each of the measuring parts 110, 120, and 130. In addition, the measurement timing may be allowed to be set as appropriate according to the condition of the patient P. In accordance with an aspect, each of the measuring parts 110, 120, and 130 may not be formed by a wearable apparatus.

The congestion measuring part 110 in the present exemplary embodiment includes a pulmonary congestion measuring part 111 capable of measuring an amount of pulmonary congestion of the patient P, and a bodily congestion measuring part 112 capable of measuring an amount of bodily congestion of the patient P. The pulmonary congestion measuring part 111 is not particularly limited as long as the pulmonary congestion measuring part 111 is capable of measuring the amount of pulmonary congestion of the patient P. However, the pulmonary congestion measuring part 111 can be an apparatus capable of measuring an amount of water in a lung of the patient P by using, for example, a thoracic impedance, ultrasonic waves, a microphone, a percutaneous arterial oxygen saturation, a local tissue oxygen saturation, or the like. The bodily congestion measuring part 112 is not particularly limited as long as the bodily congestion measuring part 112 is capable of measuring the amount of bodily congestion of the patient P. However, the bodily congestion measuring part 112 can be an apparatus capable of measuring an amount of swelling of a limb by, for example, measuring the perimeter of the limb (leg in FIG. 1) of the patient P or the bio-impedance of the limb of the patient P.

The blood flow rate measuring part 120 in the present exemplary embodiment, for example, is a temperature sensor capable of measuring a change in body surface temperature (coldness of limbs) which change accompanies a change in the blood flow rate of the limb (leg) of the patient P. However, the blood flow rate measuring part 120 is not particularly limited as long as the blood flow rate measuring part 120 is capable of measuring the blood flow rate of the patient P directly or indirectly. For example, the blood flow rate measuring part 120 may be an apparatus such as a camera capable of measuring a change in color which change accompanies a change in amount of oxygen (change in blood flow rate) in the limb of the patient P. In addition, the blood flow rate measuring part 120 may measure both of the temperature and the color of the limb of the patient P. In addition, the blood flow rate measuring part 120 may measure the blood flow rate of another body part such as the trunk (i.e., torso) of the patient P instead of the limb of the patient P.

The pump function measuring part 130 in the present exemplary embodiment includes a heartbeat measuring part 131 capable of measuring the heart rate of the patient P, and an exercise amount measuring part 132 capable of measuring an amount of exercise taken by motion of the patient P. The heartbeat measuring part 131 can be an apparatus capable of measuring the heart rate, for example, an electrocardiograph or the like. The exercise amount measuring part 132 is not particularly limited as long as the exercise amount measuring part 132 is capable of measuring the amount of exercise taken by motion of the patient P. However, the exercise amount measuring part 132 can be an apparatus such as, for example, an acceleration sensor which detects the motion of the patient P. In accordance with an aspect, while FIG. 1 represents a case where the heartbeat measuring part 131 and the exercise amount measuring part 132 are attached to the chest of the patient P, the attachment positions of the heartbeat measuring part 131 and the exercise amount measuring part 132 are not particularly limited as long as the heartbeat measuring part 131 and the exercise amount measuring part 132 can measure the heart rate and the amount of exercise of the patient P. For example, the heartbeat measuring part 131 and the exercise amount measuring part 132 may be attached to a leg of the patient P.

The control unit 140 may be connected to each of the measuring parts 110, 120, and 130 via a wireless communication network (indicated by broken lines in the figure). The control unit 140 controls measurement operation of each of the measuring parts 110, 120, and 130, obtains measurement data from each of the measuring parts 110, 120, and 130, and transmits the measurement data to the server 200.

### Server

As depicted in FIG. 3, the server 200 includes a central processing unit (CPU) 210, a storage unit 220, an input-output interface (I/F) 230, a communicating unit 240, and a reading unit 250. The CPU 210, the storage unit 220, the input-output I/F 230, the communicating unit 240, and the reading unit 250 are connected to a bus 260, and mutually exchange data or the like via the bus 260. Each part will be described in the following.

The CPU 210 performs control of each part, various kinds of arithmetic processing, and the like according to various kinds of programs stored in the storage unit 220.

The storage unit 220 includes a read only memory (ROM) storing various kinds of programs and various kinds of data, a random access memory (RAM) temporarily storing a program and data as a work area, a hard disk storing various kinds of programs including an operating system and various kinds of data, or the like. The storage unit 220 stores various kinds of programs such as a diagnostic assistance program and various kinds of data.

The communicating unit 240 is an interface for communicating with the measuring unit 100, the operating terminals 310 and 320 of the respective doctors A and B, and the like.

The reading unit 250 reads the diagnostic assistance program or the like recorded on a computer readable recording medium MD (see FIG. 1). Though not particularly limited, the computer readable recording medium MD can include, for example, an optical disk such as a compact disc (CD)-ROM or a digital versatile disc (DVD)-ROM, a universal serial bus (USB) memory, or a secure digital (SD) memory card. Though not particularly limited, the reading unit 250 can include, for example, a CD-ROM drive, a DVD-ROM drive, or the like.

Main functions of the CPU 210 will next be described.

The CPU 210 functions as a data obtaining section 211, an initial value setting section 212, a data processing section 213, and a display control section 218 as depicted in FIG. 4 by executing the diagnostic assistance program stored in the storage unit 220. Each part will be described in the following.

The data obtaining section 211 will first be described.

As depicted in FIG. 5A, the data obtaining section 211 in the present exemplary embodiment obtains, from the measuring unit 100, measurement data D1 on an amount of congestion in at least a part of the body of the patient P (which measurement data will hereinafter be referred to simply as “measurement data D1 on the amount of congestion”), measurement data D2 on the parameter related to the blood flow rate of the patient P, and measurement data D3 related to the pump function of the heart of the patient P.

The measurement data D1 on the amount of congestion in the present embodiment can include measurement data D11 on the amount of pulmonary congestion and measurement data D12 on the amount of bodily congestion.

The measurement data D2 on the parameter related to the blood flow rate in the present embodiment can include measurement data on the temperature of a limb. In accordance with an exemplary embodiment, the measurement data D2 on the parameter related to the blood flow rate will hereinafter be referred to also as the “measurement data D2 on the temperature of the limb.”

The measurement data D3 related to the pump function of the heart in the present embodiment can include measurement data D31 on the heart rate and measurement data D32 on the amount of exercise.

The data obtaining section 211 obtains the measurement data D1 on the amount of congestion, the measurement data D2 on the temperature of the limb, and the measurement data D3 related to the pump function of the heart in time series (i.e., a series of data points indexed in time order) from the measuring unit 100. The measurement data D1 on the amount of congestion, the measurement data D2 on the temperature of the limb, and the measurement data D3 related to the pump function of the heart, which are obtained in time series, are stored in the storage unit 220 in a state of being associated with each measurement time, as depicted in FIG. 5A.

The initial value setting section 212 will next be described.

The initial value setting section 212 in the present embodiment instructs a user to specify an initial value of the amount of congestion according to a degree of congestion of the patient P on a day that the measuring unit 100 starts measurement. The initial value setting section 212 sets the initial value of the amount of congestion to the value specified by the user.

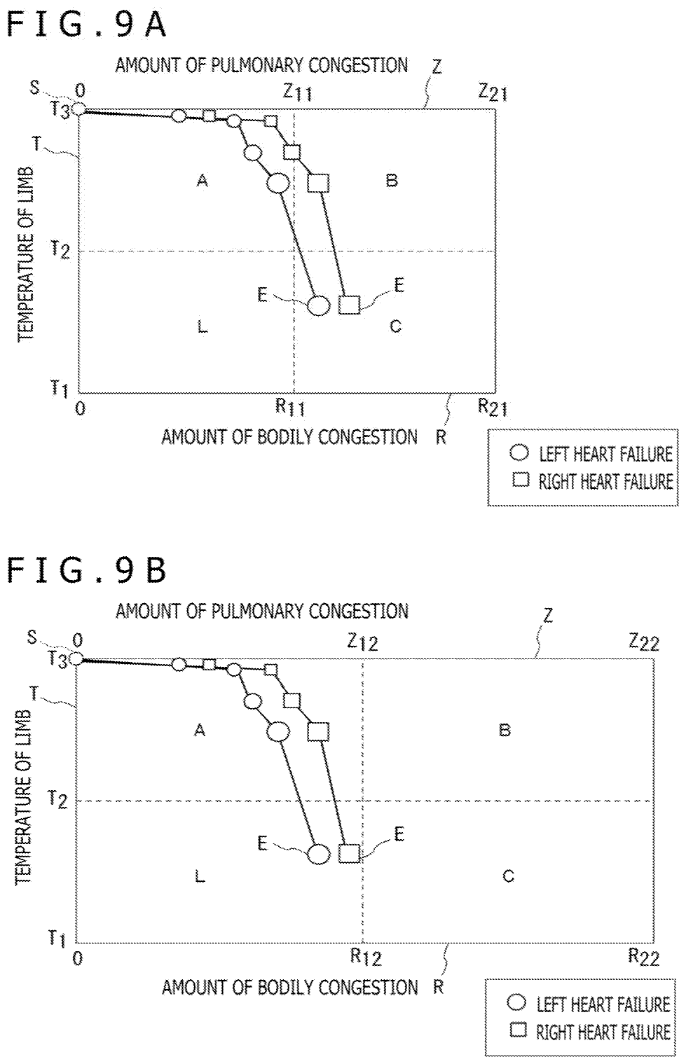

Specifically, in a case where the measuring unit 100 performs measurement from a day that the patient P is discharged from a medical institution to which the medical specialist A in heart failure belongs (which day will hereinafter be referred to simply as a “day of discharge from the hospital”), for example, the doctor A as the user specifies zero as the initial values of the amount of pulmonary congestion and the amount of bodily congestion when the pulmonary congestion and the bodily congestion of the patient P are completely cured on the day of discharge from the hospital. Thus, as depicted in FIG. 6A, in a graph to be described later, a first point S in time series is plotted at the position of zero as an amount of congestion. In addition, when the pulmonary congestion and the bodily congestion of the patient P are not sufficiently cured on the day of discharge from the hospital, for example, the doctor A as the user specifies respective threshold values Z1 and R1 (see FIG. 6B) of the amount of pulmonary congestion and the amount of bodily congestion, which threshold values will be described later, as the initial values of the amount of pulmonary congestion and the amount of bodily congestion, respectively. Thus, in the graph to be described later and shown in FIGS. 6A, 6B, 9A, and 9B, the first point S in time series is plotted at the position of the threshold values Z1 and R1 of the amount of pulmonary congestion and the amount of bodily congestion. In accordance with an aspect, while FIG. 6B depicts a case where both of the pulmonary congestion and the bodily congestion are not sufficiently cured, the doctor A can specify zero as the initial value of the amount of pulmonary congestion and specify the threshold value R1 of the amount of bodily congestion as the initial value of the amount of bodily congestion when the pulmonary congestion is cured and the bodily congestion is not sufficiently cured. In addition, when the pulmonary congestion is not sufficiently cured but the bodily congestion is cured, the doctor A can specify the threshold value Z1 as the initial value of the amount of pulmonary congestion and specify zero as the initial value of the amount of bodily congestion. In addition, the method of setting the initial values of the amounts of congestion is not limited to the foregoing. For example, according to the degrees of congestion of the patient on the day of discharge from the hospital, the doctor A as the user may freely specify a value in a range from a minimum value (zero in the present embodiment) to a maximum value Z2 or R2 on the axes of abscissas (i.e., horizontal or x-axis) Z and R of the graph to be described later.

The data processing section 213 will next be described.

The data processing section 213 preprocesses each piece of the measurement data D1, D2, and D3 before the display control section 218 to be described later displays the graph.

As depicted in FIG. 5B, the data processing section 213 calculates an average value of the measurement data D1 on the amount of congestion which measurement data is measured in predetermined timing (for example, at intervals of one minute to one hour) on a day that the measuring unit 100 starts measurement (on the day of discharge from the hospital). The calculated value will hereinafter be referred to simply as an “initial average value of the measurement data D1 on the amount of congestion.” Next, the data processing section 213 calculates a value obtained by subtracting the initial average value of the measurement data D1 on the amount of congestion from the measurement data D1 on the amount of congestion which measurement data is measured after the day that the measuring unit 100 starts measurement (after discharge from the hospital) and adding the initial value of the amount of congestion which initial value is set by the initial value setting section 212. The calculated value will hereinafter be referred to simply as an “offset value of the measurement data D1 on the amount of congestion.” Next, the data processing section 213 calculates an average value of the offset value of the measurement data D1 on the amount of congestion in each predetermined period (for example, one day). The calculated value will hereinafter be referred to as an “average value of the measurement data D1 on the amount of congestion (an average value of the measurement data D11 on the amount of pulmonary congestion and an average value of the measurement data D12 on the amount of bodily congestion).” Thus, the average value of the measurement data D1 on the amount of congestion represents an amount of change from the initial value of the amount of congestion which initial value is specified by the doctor.

The data processing section 213 calculates an average value of the measurement data D2 on the temperature of the limb which measurement data is measured in each predetermined period (for example, one day) (the calculated value will hereinafter be referred to simply as an “average value of the measurement data D2 on the temperature of the limb”).

The data processing section 213 calculates an average value of the measurement data D3 related to the pump function which measurement data is measured in each predetermined period (for example, one day) (the calculated value will hereinafter be referred to simply as an “average value of the measurement data D3 related to the pump function”). The data processing section 213 calculates the following Equation (1) using the average value of the measurement data D3 related to the pump function and thereby evaluates the degree of the pump function of the heart of the patient in each predetermined period (for example, one day).

Degree of Pump Function of Heart=Heart Rate/Amount of Exercise Equation (1)

It is to be noted that the method of preprocessing each piece of the measurement data D1, D2, and D3 by the data processing section 213 is not limited to the above. For example, instead of calculating the average value of each piece of the measurement data D1, D2, and D3 measured in each predetermined period, the data processing section 213 may calculate a median value, a minimum value, a maximum value, or the like of each piece of the measurement data D1, D2, and D3 measured in each predetermined period. The display control section 218 to be described later may then plot, in the graph, the median value, the minimum value, the maximum value, or the like of each piece of the measurement data D1, D2, and D3.

The display control section 218 will next be described.

The display control section 218 functions as a plotting section 214, a threshold value display section 217, an axis setting section 215, and an output section 216. Each part will be described in detail in the following.

As depicted in FIG. 6A and FIG. 6B, the plotting section 214 generates a graph in which a first axis of abscissas Z indicates the amount of pulmonary congestion, a second axis of abscissas R indicates the amount of bodily congestion, and an axis of ordinates T indicates the temperature of the limb. The plotting section 214 plots, on the generated graph, a first point (represented by an outlined circle in the figures) corresponding to the average value of the measurement data D11 on the amount of pulmonary congestion and the average value of the measurement data D2 on the temperature of the limb. In addition, the plotting section 214 plots, in the graph, a second point (represented by an outlined quadrangle in the figures) corresponding to the average value of the measurement data D12 on the amount of bodily congestion and the average value of the measurement data D2 on the temperature of the limb. The pulmonary congestion is caused by a left heart failure. Thus, the first point will hereinafter be referred to as a “point of the left heart failure.” In addition, the amount of bodily congestion is caused by a right heart failure. Thus, the second point will hereinafter be referred to as a “point of the right heart failure.”

The plotting section 214 plots the point of the left heart failure and the point of the right heart failure in time series. The doctors A and B as users can thereby rather easily grasp tendencies for the point of the left heart failure and the point of the right heart failure to change from the first point S to latest points E in time series, or the like. In accordance with an exemplary embodiment, the plotting section 214 performs the plotting such that the amount of congestion of the first point S in time series is the initial value of the amount of congestion which initial value is set by the initial value setting section 212.

The plotting section 214 changes display of the plotted point of the left heart failure and the plotted point of the right heart failure according to the degree of the pump function of the heart of the patient P. FIG. 6A and FIG. 6B depict a mode in which the plotting section 214 performs the plotting such that the larger the value of Equation (1) (the more the pump function of the heart is degraded), the larger each point. However, the method by which the plotting section 214 changes the display of the points is not particularly limited as long as the users of the diagnostic assistance system 10 can grasp the degree of the pump function of the heart. The method by which the plotting section 214 changes the display of the points can include, for example, a method of changing the shades of colors of the plotted points, a method of changing the colors of the plotted points, a method of changing the shapes of the plotted points, and the like.

The threshold value display section 217 will next be described.

The threshold value display section 217 displays a threshold value of the amount of congestion (the threshold value Z1 of the amount of pulmonary congestion and the threshold value R1 of the amount of bodily congestion) and a threshold value T2 of the temperature of the limb in the graph plotted by the plotting section 214. The threshold value display section 217 in the present embodiment displays the threshold value of the amount of congestion in the graph by a line drawn in a direction orthogonal to the axes of abscissas R and Z so as to pass through the threshold value of the amount of congestion (the threshold value Z1 of the amount of pulmonary congestion and the threshold value R1 of the amount of bodily congestion) in the graph. In addition, the threshold value display section 217 in the present embodiment displays the threshold value of the temperature of the limb in the graph by a line drawn in a direction orthogonal to the axis of ordinates T so as to pass through the threshold value T2 of the temperature of the limb in the graph. The graph is consequently divided into four areas. The first area is an area in which the amounts of congestion are smaller than the threshold values Z1 and R1 and in which the temperature of the limb is larger than the threshold value T2 (which area will hereinafter be referred to as an “A-area”). The A-area corresponds to the Warm & Dry area in the Nohria-Stevenson classification. The second area is an area in which the amounts of congestion are larger than the threshold values Z1 and R1 and in which the temperature of the limb is larger than the threshold value T2 (which area will hereinafter be referred to as a “B-area”). The B-area corresponds to the Warm & Wet area in the Nohria-Stevenson classification. The third area is an area in which the amounts of congestion are smaller than the threshold values Z1 and R1 and in which the temperature of the limb is smaller than the threshold value T2 (which area will hereinafter be referred to as an “L-area”). The L-area corresponds to the Cold & Dry area in the Nohria-Stevenson classification. The fourth area is an area in which the amounts of congestion are larger than the threshold values Z1 and R1 and in which the temperature of the limb is smaller than the threshold value T2 (which area will hereinafter be referred to as a “C-area”). The C-area corresponds to the Cold & Wet area in the Nohria-Stevenson classification. In accordance with an exemplary embodiment, each of the threshold values Z1, R1, and T2 can be set to be a value exceeding a predetermined exacerbation level. However, the method by which the threshold value display section 217 displays each threshold value in the graph is not particularly limited as long as the users can grasp each threshold value. For example, the threshold value display section 217 may display each threshold value in the graph by displaying a mark indicating the threshold value in a part corresponding to each threshold value on the axes of abscissas R and Z and the axis of ordinates T in the graph.

The axis setting section 215 (corresponding to a “second axis setting section”) will next be described.

The axis setting section 215 in the present embodiment sets a range of the axis of ordinates T (a maximum value T3 and a minimum value T1) on the basis of the measurement data D2 on the temperature of the limb. The axis setting section 215 in the present embodiment calculates an average value of the measurement data D2 on the temperature of the limb which measurement data is obtained in a predetermined timing (for example, at intervals of one minute to one hour) on the day that the measuring unit 100 starts measurement (on the day of discharge from the hospital) (the calculated value will hereinafter be referred to simply as an “initial average value of the measurement data D2 on the temperature of the limb”). The axis setting section 215 sets the axis of ordinates T such that the initial average value of the measurement data D2 on the temperature of the limb is the maximum value T3 of the axis of ordinates T and a value obtained by subtracting a predetermined temperature (for example, twice a difference between the maximum value T3 and the threshold value T2) from the maximum value T3 is the minimum value T1 of the axis of ordinates T. However, the range of the axis of ordinates T is not limited to the above. For example, the minimum value T1 of the axis of ordinates T may not be the value obtained by subtracting twice the difference between the maximum value T3 and the threshold value T2 from the maximum value T3. In addition, the measurement data D2 on the temperature of the limb which measurement data is used for the axis setting section 215 to set the axis of ordinates is not limited to the initial average value of the measurement data D2 on the temperature of the limb. For example, the axis setting section 215 may set the range of the axis of ordinates T such that a maximum value of the measurement data D2 in time series is the maximum value T3 of the axis of ordinates T and a minimum value of the measurement data D2 on the temperature of the limb in time series is the minimum value T1.

In accordance with an aspect, in the present embodiment, the plotting section 214 plots the graph such that ranges of the first axis of abscissas Z and the second axis of abscissas R are fixed ranges irrespective of the patient P. In accordance with an aspect, FIGS. 6A and 6B depict a mode in which minimum values of the ranges of the first axis of abscissas Z and the second axis of abscissas R are zero and values obtained by adding predetermined values (for example, values twice the threshold values Z1 and R1) to the minimum values are maximum values Z2 and R2 of the first axis of abscissas Z and the second axis of abscissas R. However, the ranges of the first axis of abscissas Z and the second axis of abscissas R are not limited to the above. For example, the minimum values of the first axis of abscissas Z and the second axis of abscissas R may not be zero. In addition, the maximum values of the first axis of abscissas Z and the second axis of abscissas R may not be the values obtained by adding the values twice the threshold values Z1 and R1 to the minimum values.

The output section 216 will next be described.

The output section 216 in the present embodiment displays the graph on at least one of display units 310a or 320a (see FIG. 1) of each of the operating terminals of the doctors A and B as users. In accordance with an exemplary embodiment, the output section 216 may further display the graph on a display unit 140a of the control unit 140.

### Diagnostic Assistance Method

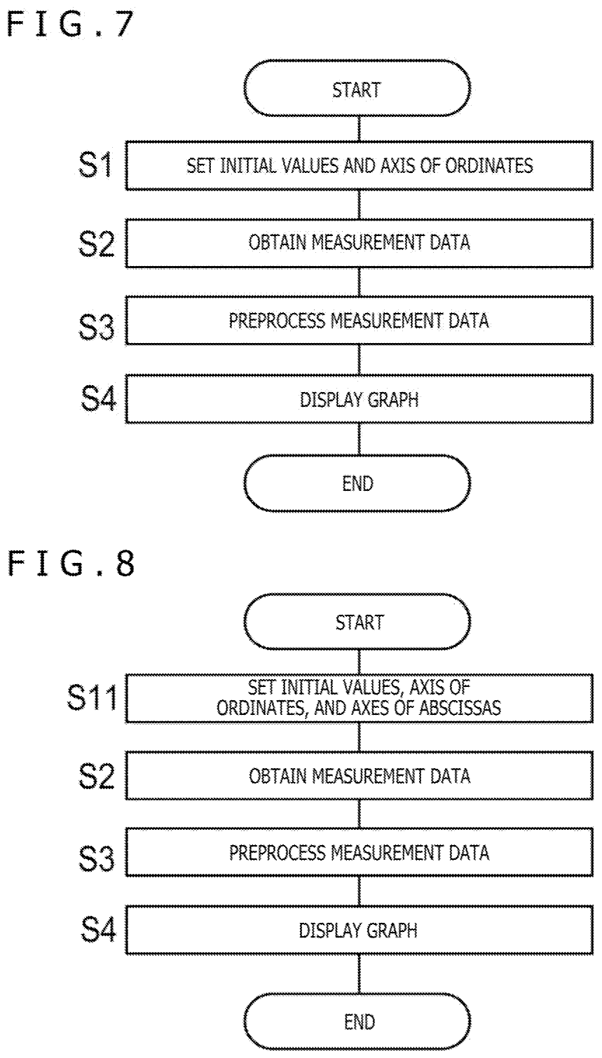

A diagnostic assistance method according to the present embodiment will next be described. FIG. 7 is a flowchart depicting the diagnostic assistance method according to the embodiment of the present disclosure. In the following, description will be made by taking, as an example, a case where the patient P is discharged from the medical institution to which the medical specialist A belongs, and the general physician B at a clinic recommends the patient P receive a treatment from the medical specialist A as appropriate according to the condition of the patient P while the general physician B observes the progress of the discharged patient P.

Generally described with reference to FIG. 7, the diagnostic assistance method according to the present embodiment sets the initial value of the amount of congestion and the axis of ordinates T of the graph (setting step S1), obtains the measurement data D1 on the amount of congestion in at least a part of the body of the patient P, the measurement data D2 on the temperature of the limb, and the measurement data D3 related to the pump function of the heart (data obtaining step S2), preprocesses the obtained measurement data D1, D2, and D3 (data processing step S3), and displays a point corresponding to the measurement data D1 on the amount of congestion and the measurement data D2 on the temperature of the limb in the graph in which the axes of abscissas Z and R indicate amounts of congestion and in which the axis of ordinates T indicates the temperature of the limb (display step S4). Each step will be described in detail in the following.

The setting step S1 will first be described. The setting step S1 is, for example, performed on a day that the patient P is discharged from the medical institution to which the medical specialist A belongs.

The patient P attaches each of the measuring parts 110, 120, and 130 of the measuring unit 100 to the body of the patient P on the day of discharge from the hospital. In accordance with an exemplary embodiment, the measuring unit 100 subsequently measures the amount of pulmonary congestion, the amount of bodily congestion, the blood flow rate, the heart rate, and the amount of exercise in predetermined timing (for example, at intervals of one minute to one hour). However, the measuring unit 100 may stop the measurement when each of the measuring parts 110, 120, and 130 is removed from the body of the patient P.

Next, the data obtaining section 211 obtains each piece of the measurement data D1, D2, and D3 measured on the day of discharge from the hospital from the measuring unit 100.

Next, the axis setting section 215 calculates an average value of the measurement data D2 on the temperature of the limb (an initial average value of the measurement data D2 on the temperature of the limb) on the day of discharge from the hospital by using the measurement data D2 on the temperature of the limb which measurement data is measured on the day of discharge from the hospital. Next, the axis setting section 215 sets the axis of ordinates T such that the initial average value of the measurement data D2 on the temperature of the limb is the maximum value T3 of the axis of ordinates T and a value obtained by subtracting a predetermined temperature (for example, twice a difference between the maximum value T3 and the threshold value T2) from the maximum value T3 is the minimum value T1 of the axis of ordinates T. The axis setting section 215 can therefore set the axis of ordinates T according to an individual difference of the patient P.

Next, the initial value setting section 212 instructs the medical specialist A to specify an initial value of the amount of congestion. The medical specialist A, for example, specifies zero as the initial value of the amount of pulmonary congestion when the pulmonary congestion of the patient P is cured completely. In addition, the medical specialist A specifies zero as the initial value of the amount of bodily congestion when the bodily congestion of the patient P is cured completely. The medical specialist A, for example, also specifies the threshold value Z1 of the amount of pulmonary congestion as the initial value of the amount of pulmonary congestion when the pulmonary congestion of the patient P is not cured. In addition, the medical specialist A specifies the threshold value R1 of the amount of bodily congestion as the initial value of the amount of bodily congestion when the bodily congestion of the patient P is not cured. The initial value setting section 212 sets the initial value of the amount of congestion (the initial value of the pulmonary congestion and the initial value of the bodily congestion) on the basis of the specified value.

In accordance with an exemplary embodiment, the order in which the setting of the initial value of the amount of congestion and the setting of the axis of ordinates T of the graph in the setting step S1 are performed is not particularly limited. For example, the setting of the initial value of the amount of congestion may be performed first, and then, the setting of the axis of ordinates T of the graph may be performed. In addition, the setting of the initial value of the amount of congestion and the setting of the axis of ordinates T of the graph may be performed simultaneously.

The data obtaining step S2 to the display step S4 will next be described.

The data obtaining step S2 to the display step S4 are, for example, performed after discharge from the hospital.

The data obtaining step S2 will first be described.

The data obtaining section 211 obtains each piece of the measurement data D1, D2, and D3 after discharge from the hospital from the measuring unit 100 in a predetermined timing. The timing in which the data obtaining section 211 obtains each piece of the measurement data D1, D2, and D3 from the measuring unit 100 is not particularly limited. However, for example, the data obtaining section 211 can obtain each piece of the measurement data D1, D2, and D3 from the measuring unit 100 once a day, in a timing in which a request to provide the graph is made from each of the doctors A and B as a user, or the like.

The data processing step S3 will next be described.

The data processing step S3 preprocesses each piece of the measurement data D1, D2, and D3.

First, as depicted in FIG. 5B, the data processing section 213 calculates an average value of the measurement data D1 on the amount of congestion (an initial average value of the measurement data D1 on the amount of congestion) which measurement data is obtained on the day of discharge from the hospital. Next, the data processing section 213 calculates a value (offset value of the measurement data D1 on the amount of congestion) obtained by subtracting the initial average value of the measurement data D1 on the amount of congestion from the measurement data D1 on the amount of congestion which measurement data is obtained after discharge from the hospital and adding the initial value of the amount of congestion which initial value is set by the initial value setting section 212. Next, the data processing section 213 calculates an average value of the offset value of the measurement data D1 on the amount of congestion (average value of the measurement data D1 on the amount of congestion) which measurement data is obtained in each predetermined period (for example, one day).

Next, the data processing section 213 calculates an average value of the measurement data D2 on the temperature of the limb which measurement data is obtained in each predetermined period (for example, one day).

Next, the data processing section 213 calculates an average value of the measurement data D3 related to the pump function which measurement data is obtained in each predetermined period (for example, one day). The data processing section 213 calculates the above-described Equation (1) using the average value of the measurement data D3 related to the pump function and thereby evaluates the degree of the pump function of the heart of the patient in each predetermined period (for example, one day).

In accordance with an exemplary embodiment, the data processing step S3 may be performed in each predetermined period (for example, one day), or may be performed in a timing in which a request to provide the graph is made from a user of the system. In addition, the order in which the preprocessing of each piece of the measurement data D1, D2, and D3 is performed is not limited to the above. For example, the preprocessing of the measurement data D2 on the temperature of the limb may be performed first, or the preprocessing of the measurement data D3 related to the pump function may be performed first. In addition, the preprocessing of each piece of the measurement data D1, D2, and D3 may be performed simultaneously.

The display step S4 will next be described.

As depicted in FIG. 6A and FIG. 6B, the plotting section 214 generates a graph in which a first axis of abscissas Z indicates the amount of pulmonary congestion, a second axis of abscissas R indicates the amount of bodily congestion, and an axis of ordinates T indicates the temperature of the limb. The plotting section 214 plots, in the graph, a point of the left heart failure which point corresponds to the average value of the measurement data D11 on the amount of pulmonary congestion and the average value of the measurement data D2 on the temperature of the limb, the average values being calculated in step S3, and a point of the right heart failure which point corresponds to the average value of the measurement data D12 on the amount of bodily congestion and the average value of the measurement data D2 on the temperature of the limb, the average values being calculated in step S3. The doctors A and B can therefore diagnose the patient P while cooperating with each other using the graph as a common index. Further, for example, even the general physician B who has less experience in diagnosing heart failure than the medical specialist A can diagnose the patient P rather easily while referring to the graph. In addition, the doctors A and B as users can obtain both of information effective in diagnosing the left heart failure of the patient P and information effective in diagnosing the right heart failure of the patient P. Therefore, the doctors A and B as users can rather easily grasp that a heart is in a failure state.

In accordance with an exemplary embodiment, at this time, the threshold value display section 217 displays, in the graph, the threshold value of the amount of congestion (the threshold value Z1 of the amount of pulmonary congestion and the threshold value R1 of the amount of bodily congestion) and the threshold value T2 of the temperature of the limb. The doctors A and B can therefore rather easily classify the disease state of the patient P on the basis of the Nohria-Stevenson classification. In addition, at this time, the plotting section 214 changes display of the plotted point of the left heart failure and the plotted point of the right heart failure according to the degree of the pump function of the heart of the patient P which degree is calculated in step S3. The doctors A and B as users can therefore rather easily grasp the degree of the pump function of the patient P.

Next, the output section 216 displays the graph on at least one of the display units 310a or 320a of each of the operating terminals of the doctors A and B as users. In accordance with an exemplary embodiment, the output section 216 may further display the graph on the display unit 140a of the control unit 140.

In accordance with an exemplary embodiment, the display step S4 may be performed in each predetermined period (for example, one day), or may be performed in timing in which a request to provide the graph is made from a user of the system.

The diagnostic assistance method according to the present embodiment has been described above. However, the diagnostic assistance method is not limited to the above. For example, the measuring unit 100 may not start measurement from the day of discharge from the hospital on which day the patient P is discharged from the medical institution to which the medical specialist A belongs. The measuring unit 100 may start measurement from a day that the patient P visits the clinic to which the doctor B belongs. In this case, each of steps S1 to S4 can be performed with the clinic visit day set as a day that the measuring unit 100 starts measurement. In addition, steps S1 to S4 (or steps S2 to S4) may be repeatedly performed in a predetermined timing.

As described above, the diagnostic assistance system 10 according to the foregoing embodiment is a diagnostic assistance system that assists in diagnosis of heart failure. The diagnostic assistance system 10 can include the data obtaining section 211 that obtains the measurement data D1 on the amount of congestion in at least a part of the body of the patient P and the measurement data D2 on the parameter related to the blood flow rate of the patient P, and the display control section 218 that displays a graph in which an axis of abscissas indicates the amount of congestion and an axis of ordinates indicates the parameter related to the blood flow rate on the display units 310a and 320a. The display control section 218 displays, in the graph, a point corresponding to the measurement data D1 on the amount of congestion and the measurement data D2 on the parameter related to the blood flow rate.

According to the diagnostic assistance system 10 described above, the doctors A and B as users can use the graph displaying the point corresponding to the measurement data D1 on the amount of congestion in at least a part of the body of the patient P and the measurement data D2 on the parameter related to the blood flow rate of the patient P as a common index in diagnosis of heart failure on the basis of the Nohria-Stevenson classification.

In addition, the data obtaining section 211 obtains the measurement data D1 on the amount of congestion and the measurement data D2 on the parameter related to the blood flow rate in time series, and the display control section 218 displays, in the graph, points corresponding to the measurement data D1 on the amount of congestion and the measurement data D2 on the parameter related to the blood flow rate in time series. The doctors A and B as users, or the like can therefore grasp tendencies of changes in the amount of congestion and the blood flow rate.

In addition, the display control section 218 can include the threshold value display section 217 that displays, in the graph, the threshold values Z1 and R1 of amounts of congestion and the threshold value T2 of the parameter related to the blood flow rate. The doctors A and B or the like can therefore rather easily classify the disease state of the patient P on the basis of the Nohria-Stevenson classification.

In addition, the measurement data D1 on the amount of congestion can include the measurement data D11 on the amount of pulmonary congestion of the patient P and the measurement data D12 on the amount of bodily congestion of the patient P, and the display control section 218 displays, in the graph, the first point corresponding to the measurement data D11 on the amount of pulmonary congestion and the measurement data D2 on the parameter related to the blood flow rate and the second point corresponding to the measurement data D12 on the amount of bodily congestion and the measurement data D2 on the parameter related to the blood flow rate. The doctors A and B as users can therefore obtain both of information effective in diagnosing the left heart failure of the patient P and information effective in diagnosing the right heart failure of the patient P. The doctors A and B as users can therefore rather easily grasp when a heart is in a failure state.

In addition, the parameter related to the blood flow rate can include the temperature of a limb of the patient P and/or the color of the limb of the patient P. The doctors A and B as users can therefore grasp the blood flow rate of the limb of the patient P via measurement data on the temperature of the limb of the patient and/or the color of the limb of the patient.

In addition, the data obtaining section 211 further obtains the measurement data D3 related to the pump function of the heart of the patient P, and the display control section 218 changes the display of the point according to the degree of the pump function. The doctors A and B as users can therefore rather easily grasp the degree of the pump function of the heart of the patient P. The doctors A and B can therefore make the diagnosis rather easy.

In addition, the display control section 218 in the diagnostic assistance system 10 can include the axis setting section 215 that sets the range of the axis of ordinates T on the basis of the measurement data on the parameter related to the blood flow rate of the patient P. The range of the axis of ordinates T can therefore be set to be a range according to an individual difference of the patient.

In addition, the diagnostic assistance method according to the foregoing embodiment is a method of assisting in diagnosis of heart failure. The diagnostic assistance method obtains the measurement data D1 on the amount of congestion in at least a part of the body of the patient P and the measurement data D2 on the parameter related to the blood flow rate of the patient P, and displays a point corresponding to the measurement data D1 on the amount of congestion and the measurement data D2 on the parameter related to the blood flow rate in the graph in which the axes of abscissas Z and R indicate amounts of congestion and in which the axis of ordinates T indicates the parameter related to the blood flow rate.

In addition, the diagnostic assistance program according to the foregoing embodiment is a diagnostic assistance program for assisting in diagnosis of heart failure. The diagnostic assistance program performs obtaining the measurement data D1 on the amount of congestion in at least a part of the body of the patient P and the measurement data D2 on the parameter related to the blood flow rate of the patient P, and displaying a point corresponding to the measurement data D1 on the amount of congestion and the measurement data D2 on the parameter related to the blood flow rate in the graph in which the axes of abscissas Z and R indicate amounts of congestion and the axis of ordinates T indicates the parameter related to the blood flow rate.

According to the diagnostic assistance method and the diagnostic assistance program described above, the doctors A and B as users can use the graph displaying the point corresponding to the measurement data D1 on the amount of congestion in at least a part of the body of the patient P and the measurement data D2 on the parameter related to the blood flow rate of the patient P as a common index in diagnosis of heart failure on the basis of the Nohria-Stevenson classification.

FIGS. 8 to 9B are diagrams of assistance in explaining a diagnostic assistance system 10 and a diagnostic assistance method according to a modification of the exemplary embodiment.

The diagnostic assistance system 10 according to the modification is different from the foregoing embodiment in that the axis setting section 215 (corresponding to a “first axis setting section”) sets the ranges of the first axis of abscissas Z and the second axis of abscissas R on the basis of an allowable level of the amount of congestion of the patient P which allowable level is set by the doctor A. That is, the diagnostic assistance method according to the modification is different from the foregoing embodiment in a setting step S11. The setting step as a difference will be described in the following. In addition, configurations similar to those of the foregoing embodiment are identified by the same reference signs.

As in the foregoing embodiment, the setting step S11 may be, for example, performed on the day that the patient P is discharged from the medical institution to which the medical specialist A belongs.

The patient P attaches each of the measuring parts 110, 120, and 130 of the measuring unit 100 to the body of the patient P on the day of discharge from the hospital. In accordance with an exemplary embodiment, the measuring unit 100 subsequently measures the amount of pulmonary congestion, the amount of bodily congestion, the blood flow rate, the heart rate, and the amount of exercise in a predetermined timing (for example, at intervals of one minute to one hour). However, the measuring unit 100 may stop the measurement when each of the measuring parts 110, 120, and 130 is removed from the body of the patient P.

Next, the data obtaining section 211 obtains each piece of the measurement data D1, D2, and D3 measured on the day of discharge from the hospital from the measuring unit 100.

Next, the axis setting section 215 calculates an average value of the measurement data D2 on the temperature of the limb (an initial average value of the measurement data D2 on the temperature of the limb) on the day of discharge from the hospital by using the measurement data D2 on the temperature of the limb which measurement data is measured on the day of discharge from the hospital. Next, the axis setting section 215 sets the axis of ordinates T such that the initial average value of the measurement data D2 on the temperature of the limb is the maximum value T3 of the axis of ordinates T and a value obtained by subtracting a predetermined temperature (for example, twice a difference between the maximum value T3 and the threshold value T2) from the maximum value T3 is the minimum value T1 of the axis of ordinates T.

Next, the axis setting section 215 instructs the doctor A to input an allowable level of the amount of congestion of the patient P (for example, three levels of “high,” “standard,” and “low”). The axis setting section 215 sets the maximum values and threshold values of the first axis of abscissas Z and the second axis of abscissas R according to the input allowable level of the amount of congestion of the patient P. For example, when the allowable level of the amount of congestion of the patient P is lower than a standard, the doctor A as a user inputs “low” as the allowable level of the amount of congestion of the patient P. On the basis of the input allowable level of congestion, as depicted in FIG. 9A, the axis setting section 215 sets the maximum values of the first axis of abscissas Z and the second axis of abscissas R at values Z21 and R21 smaller than the standard, and sets half values Z11 and R11 of the maximum values Z21 and R21 as threshold values. In addition, for example, when the allowable level of the amount of congestion of the patient P is higher than the standard, the doctor A as a user inputs “high” as the allowable level of the amount of congestion of the patient. On the basis of the input allowable level of congestion, as depicted in FIG. 9B, the axis setting section 215 sets the maximum values of the first axis of abscissas Z and the second axis of abscissas R at values Z22 and R22 larger than the standard, and sets half values Z12 and R12 of the maximum values Z22 and R22 as threshold values. However, the ranges of the first axis of abscissas Z and the second axis of abscissas R are not limited to the above. For example, minimum values of the first axis of abscissas Z and the second axis of abscissas R may not be zero. In addition, the threshold values of the first axis of abscissas Z and the second axis of abscissas R may not be half the maximum values. In addition, the allowable level of the amount of congestion of the patient P may be divided, for example, into two levels or four levels instead of three levels.

Next, the initial value setting section 212 instructs the medical specialist A to specify an initial value of the amount of congestion. The medical specialist A, for example, specifies zero as the initial value of the amount of congestion when the bodily congestion and the pulmonary congestion of the patient P are cured completely. In addition, the medical specialist A, for example, specifies the threshold values Z1 and R1 of amounts of congestion as the initial value of the amount of congestion when the bodily congestion and the pulmonary congestion of the patient P are not cured. The initial value setting section 212 next sets the initial value of the amount of congestion on the basis of the specified value.

In accordance with an exemplary embodiment, the order in which the setting of the axis of ordinates T, the setting of the axes of abscissas Z and R, and the setting of the initial value of the amount of congestion are performed is not limited to the above. For example, the setting of the initial value of the amount of congestion may be performed first, and then, the setting of the axis of ordinates T and the setting of the axes of abscissas Z and R in the graph may be performed. In addition, the setting of the axes of abscissas Z and R and the setting of the initial value of the amount of congestion may be performed simultaneously.

As described above, the display control section 218 in the diagnostic assistance system 10 according to the modification of the exemplary embodiment can include the axis setting section 215 that sets the ranges of the axes of abscissas Z and R and/or the threshold value of the amount of congestion on the basis of the allowable level of the amount of congestion of the patient P which allowable level is set by the doctor A as a user. The ranges of the axes of abscissas Z and R can therefore be set to be a range according to individual differences of each of the patients.

The present disclosure has been described above through exemplary embodiments and modifications of the exemplary embodiments. However, the present disclosure is not limited to only each of the described configurations, but can be modified as appropriate on the basis of the description of claims.

For example, sections and methods for performing various kinds of processing in the diagnostic assistance system may be implemented by either a dedicated hardware circuit or a programmed computer. In addition, the diagnostic assistance program may be provided online via a network such as the Internet.

In addition, the diagnostic assistance system may include only the server 200 in the foregoing embodiment, and may be used in combination with another measuring device capable of measuring the amount of congestion in at least a part of the body of the patient and the parameter related to the blood flow rate of the patient (for example, the diagnostic assistance system may not include the measuring unit 100).

In addition, while each of the configurations of the server 200 has been described as being implemented as one device in the foregoing embodiment, the configurations of the apparatus are not limited to this. For example, the server 200 may include a plurality of servers and may virtually include a large number of servers installed at remote places as cloud servers.

The CPU of the control unit of the measuring unit may also function as the data obtaining section, the display control section, and the like. In addition, for example, the diagnostic assistance program may be installed on the operating terminal of a user, and a CPU of the operating terminal of the user may function as the data obtaining section, the display control section, and the like.

In addition, the diagnostic assistance system may be configured to obtain the measurement data on only either the amount of pulmonary congestion or the amount of bodily congestion, and display the measurement data on only either the amount of pulmonary congestion or the amount of bodily congestion in the graph. In addition, the diagnostic assistance system may be configured to obtain the measurement data on both of the amount of pulmonary congestion and the amount of bodily congestion, and display only either the amount of pulmonary congestion or the amount of bodily congestion in the graph.

In accordance with an exemplary embodiment, the diagnostic assistance system may not display the measurement data in time series.

In addition, the measurement data may not be preprocessed before being displayed.

In accordance with an exemplary embodiment, the measurement data related to the pump function of the heart may not be obtained. In addition, the method of evaluating the pump function of the heart is not limited to the above-described method of performing the evaluation on the basis of the heart rate and the amount of exercise. For example, the pump function of the heart may be evaluated on the basis of a respiration rate, a respiration pattern, fluctuations in the heart rate, or the like.

In addition, the display control section may display, in the graph, only one of the threshold value of the amount of congestion and the threshold value of the parameter related to the blood flow rate.

In addition, it suffices for users of the diagnostic assistance system to be persons who need the graph, and users of the diagnostic assistance system are not limited to only doctors. For example, users of the diagnostic assistance system may include not only the doctors but also the patient himself/herself.

In addition, the diagnostic assistance system is not limited to being used for the medical specialist in heart failure and the general physician at the clinic to examine the patient in cooperation with each other as in the foregoing embodiment. For example, the diagnostic assistance system may be used for a plurality of medical specialists (or general physicians) belonging to a same medical institution to examine one patient in cooperation with each other. In addition, the diagnostic assistance system is not limited to being used to observe progress after the patient who has once suffered from heart failure is discharged from the hospital (management of a prognosis). For example, the diagnostic assistance system may be used when a patient having a strong possibility of suffering from heart failure is diagnosed.

The detailed description above describes embodiments of a diagnostic assistance system, a diagnostic assistance method, and a diagnostic assistance program configured to assist in diagnosing heart failure. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A diagnostic assistance system for assisting in diagnosis of heart failure, the diagnostic assistance system comprising:
a data obtaining section configured to obtain measurement data on an amount of congestion in at least a part of a body of a patient and measurement data on a parameter related to a blood flow rate of the patient;
a display control section configured to display, on a display unit, a graph in which a first axis indicates the amount of congestion and a second axis indicates the parameter related to the blood flow rate; and
the display control section is configured to display, in the graph, a point corresponding to the measurement data on the amount of congestion and the measurement data on the parameter related to the blood flow rate.

2. The diagnostic assistance system according to claim 1, wherein
the data obtaining section is configured to obtain the measurement data on the amount of congestion and the measurement data on the parameter related to the blood flow rate in time series; and
the display control section is configured to display, in the graph, points corresponding to the measurement data on the amount of congestion and the measurement data on the parameter related to the blood flow rate in time series.

3. The diagnostic assistance system according to claim 1, wherein
the display control section includes a threshold value display section configured to display, in the graph, a threshold value of the amount of congestion and/or a threshold value of the parameter related to the blood flow rate.

4. The diagnostic assistance system according to claim 1, wherein
the measurement data on the amount of congestion includes measurement data on an amount of pulmonary congestion of the patient and measurement data on an amount of bodily congestion of the patient; and
the display control section is configured to display, in the graph, a point corresponding to the measurement data on the amount of pulmonary congestion and the measurement data on the parameter related to the blood flow rate and a point corresponding to the measurement data on the amount of bodily congestion and the measurement data on the parameter related to the blood flow rate.

5. The diagnostic assistance system according to claim 1, wherein
the parameter related to the blood flow rate includes a temperature of a limb of the patient and/or a color of the limb of the patient.

6. The diagnostic assistance system according to claim 1, wherein
the data obtaining section further obtains measurement data related to a pump function of a heart of the patient; and
the display control section is configured to change the display of the point according to a degree of the pump function of the heart of the patient.

7. The diagnostic assistance system according to claim 1, wherein
the display control section includes a first axis setting section configured to set a range of the first axis on a basis of an allowable level of the amount of congestion of the patient, the allowable level being set by a user.

8. The diagnostic assistance system according to claim 1, wherein
the display control section includes a second axis setting section configured to set a range of the second axis of the graph on a basis of the measurement data on the parameter related to the blood flow rate.

9. A diagnostic assistance method for assisting in diagnosis of heart failure, the diagnostic assistance method comprising:
obtaining measurement data on an amount of congestion in at least a part of a body of a patient and measurement data on a parameter related to a blood flow rate of the patient; and
displaying a point corresponding to the measurement data on the amount of congestion and the measurement data on the parameter related to the blood flow rate in a graph in which a first axis indicates the amount of congestion and a second axis indicates the parameter related to the blood flow rate.

10. The diagnostic assistance method according to claim 9, further comprising:
obtaining the measurement data on the amount of congestion and the measurement data on the parameter related to the blood flow rate in time series; and
displaying, in the graph, points corresponding to the measurement data on the amount of congestion and the measurement data on the parameter related to the blood flow rate in time series.

11. The diagnostic assistance method according to claim 9, further comprising:
displaying, in the graph, a threshold value of the amount of congestion and/or a threshold value of the parameter related to the blood flow rate.

12. The diagnostic assistance method according to claim 9, wherein the measurement data on the amount of congestion includes measurement data on an amount of pulmonary congestion of the patient and measurement data on an amount of bodily congestion of the patient, the method comprising:
displaying, in the graph, a point corresponding to the measurement data on the amount of pulmonary congestion and the measurement data on the parameter related to the blood flow rate and a point corresponding to the measurement data on the amount of bodily congestion and the measurement data on the parameter related to the blood flow rate.

13. The diagnostic assistance method according to claim 9, wherein the parameter related to the blood flow rate includes a temperature of a limb of the patient and/or a color of the limb of the patient.

14. The diagnostic assistance method according to claim 9, further comprising:
obtaining measurement data related to a pump function of a heart of the patient; and
changing the display of the point according to a degree of the pump function of the heart of the patient.

15. The diagnostic assistance method according to claim 9, further comprising:
setting a range of the first axis on a basis of an allowable level of the amount of congestion of the patient.

16. The diagnostic assistance method according to claim 9, further comprising:
setting a range of a second axis of the graph on a basis of the measurement data on the parameter related to the blood flow rate.

17. A non-transitory computer readable medium for assisting in diagnosis of heart failure, the non-transitory computer readable medium having instructions operable to cause one or more processors to perform operations comprising:
obtaining measurement data on an amount of congestion in at least a part of a body of a patient and measurement data on a parameter related to a blood flow rate of the patient; and
displaying a point corresponding to the measurement data on the amount of congestion and the measurement data on the parameter related to the blood flow rate in a graph in which a first axis indicates the amount of congestion and a second axis indicates the parameter related to the blood flow rate.

18. The non-transitory computer-readable medium according to claim 17, further comprising:
obtaining the measurement data on the amount of congestion and the measurement data on the parameter related to the blood flow rate in time series; and
displaying, in the graph, points corresponding to the measurement data on the amount of congestion and the measurement data on the parameter related to the blood flow rate in time series.

19. The non-transitory computer-readable medium according to claim 17, further comprising:
displaying, in the graph, a threshold value of the amount of congestion and/or a threshold value of the parameter related to the blood flow rate.

20. The non-transitory computer-readable medium according to claim 17, wherein the measurement data on the amount of congestion includes measurement data on an amount of pulmonary congestion of the patient and measurement data on an amount of bodily congestion of the patient, the operations further comprising:
displaying, in the graph, a point corresponding to the measurement data on the amount of pulmonary congestion and the measurement data on the parameter related to the blood flow rate and a point corresponding to the measurement data on the amount of bodily congestion and the measurement data on the parameter related to the blood flow rate.
